(19) United States
(12) Patent Application Publication
(10) Pub. No.: US 2026/0158117 A1
(43) Pub. Date: Jun. 11, 2026

(54) PHARMACEUTICAL COMPOSITIONS AND METHODS USING MESENCHYMAL STROMAL CELL-SECRETED LACTOFERRIN FOR TREATING VIRUS-INDUCED ACUTE LUNG INJURY

(71) Applicant: Centre for Immunology & Infection Limited, Hong Kong (HK)

(72) Inventors: Chi Wai Michael CHAN, Hong Kong (HK); Po Yee MA, Hong Kong (HK); Hiu Ha CHING, Hong Kong (HK)

(21) Appl. No.: 19/403,240

(22) Filed: Nov. 28, 2025

Related U.S. Application Data
(60) Provisional application No. 63/730,422, filed on Dec. 10, 2024.

(51) Int. Cl. A61K 38/40; A61K 31/351; A61K 35/28; A61P 31/16

(52) U.S. Cl. CPC A61K 38/40; A61K 31/351; A61K 35/28; A61P 31/16

(57) ABSTRACT

The invention relates to the therapeutic use of lactoferrin (LTF), a multifunctional glycoprotein abundantly present in mesenchymal stromal cell-derived extracellular vesicles (MSC-EVs), for treating acute lung injury (ALI) caused by respiratory viral infections. LTF restores alveolar fluid clearance, reduces alveolar protein permeability, and downregulates proinflammatory cytokines in H5N1-infected human alveolar epithelial cells. In vivo administration of LTF significantly improves survival, suppresses lung inflammation, and reduces viral load in infected mice. The therapeutic efficacy of LTF is shown to be comparable to that of MSC-EVs. This invention provides a novel, cell-free, and scalable strategy that bypasses the limitations of stem cell-based therapies while retaining potent antiviral and immunomodulatory effects. As an FDA-approved supplement with established safety, LTF may be rapidly repurposed for clinical use in respiratory diseases involving epithelial barrier dysfunction, inflammation, and viral infection, including influenza-induced ALI.

### CROSS-REFERENCE TO RELATED APPLICATIONS

The present application claims the priorities from the U.S. provisional patent application Ser. No. 63/730,422 filed Dec. 10, 2024, and the disclosure of which is incorporated herein by reference in its entirety.

### FIELD OF THE INVENTION

This invention utilizes an FDA-approved supplement lactoferrin, discovered for its potential therapeutic role due to its abundance in mesenchymal stromal cell-derived extracellular vesicles (MSC-EVs), for the treatment of respiratory diseases.

### BACKGROUND OF THE INVENTION

Acute lung injury (ALI) is a life-threatening condition characterized by severe pulmonary inflammation, disruption of the alveolar-capillary barrier, and impaired alveolar fluid clearance, often triggered by respiratory pathogens such as highly pathogenic influenza viruses. Current treatment strategies for ALI are largely supportive, including mechanical ventilation and fluid management, with limited pharmacological interventions that target the underlying inflammatory and epithelial injury mechanisms.

In recent years, cell-based therapies using mesenchymal stromal cells (MSCs) have gained attention due to their immunomodulatory and regenerative properties. However, the clinical application of MSCs faces several challenges, including the need for in vitro expansion, potential loss of cellular functionality, and safety concerns such as genetic instability or immune reactions. As an alternative, MSC-EVs, which carry functional proteins and RNAs that mediate paracrine effects, have been explored to overcome these limitations.

Among the protein cargo of MSC-EVs, lactoferrin (LTF) is a multifunctional glycoprotein known for its anti-inflammatory, antimicrobial, and antioxidant activities in various disease models, including SARS-CoV-2 and chronic obstructive pulmonary disease (COPD). However, with the small number of existing preliminary studies available and a lack of studies investigating the role of LTF in influenza infections, the therapeutic potential of LTF in highly pathogenic influenza virus infection has to be confirmed with more in-depth pre-clinical studies before its potential assessment in subsequent clinical trials.

Therefore, there is a need in the art for a safe, scalable, and cell-free therapeutic strategy capable of mitigating acute lung injury caused by respiratory viral infections. Such a strategy should ideally combine anti-inflammatory and antiviral properties, preserve alveolar epithelial barrier integrity, and be amenable to rapid clinical translation without the risks and limitations associated with cell-based therapies such as mesenchymal stromal cell transplantation.

### SUMMARY OF THE INVENTION

In view of the limitations associated with existing cell-based therapies for acute lung injury (ALI), including immunogenic risks, manufacturing complexity, and regulatory hurdles, the present invention aims to develop a safe, cell-free, and readily translatable therapeutic strategy. A key objective of the invention is to identify and validate functional bioactive components derived from MSC-EVs that can exert equivalent therapeutic effects without requiring intact cells.

The invention provides a method of treating acute lung injury caused by respiratory viral infections, comprising administering an effective amount of LTF, a multifunctional glycoprotein abundantly present in MSC-EV protein cargo. The method may further include evaluating the therapeutic efficacy of LTF in restoring alveolar fluid clearance, reducing epithelial protein leakage, downregulating inflammatory cytokines, and suppressing viral replication.

In accordance with one aspect of the present invention, the present invention provides a method of treating acute lung injury in a subject infected with a respiratory virus, comprising administering to the subject a composition comprising lactoferrin identified as a functional protein enriched in extracellular vesicles derived from umbilical cord-derived mesenchymal stromal cells (UC-MSC-EVs). The administration increases alveolar fluid clearance by at least 50% and reduces alveolar epithelial protein permeability by at least 50% relative to a non-treated control.

In accordance with one aspect of the present invention, the respiratory virus includes a highly pathogenic avian influenza virus.

In accordance with one aspect of the present invention, the highly pathogenic avian influenza virus includes A/HK/483/97 or A/HK/486/1997 H5N1 virus.

In accordance with one aspect of the present invention, the composition includes lactoferrin at a dose of about 100 to 1000 mg/kg body weight per day.

In accordance with one aspect of the present invention, the lactoferrin is selected from recombinant human lactoferrin or purified bovine lactoferrin.

In accordance with one aspect of the present invention, the composition is administered intravenously, intranasally, orally, or by inhalation.

In accordance with one aspect of the present invention, the administration reduces expression of one or more cytokines selected from the group consisting of MCP-1, IL-6, IL-8, RANTES, TNF-α, and MIP-1α.

In accordance with one aspect of the present invention, the composition inhibits attachment or internalization of the virus into alveolar epithelial cells.

In accordance with one aspect of the present invention, the composition suppresses expression of the influenza viral matrix (M) gene.

In accordance with one aspect of the present invention, the lactoferrin is formulated with one or more excipients selected from stabilizers, surfactants, or buffers suitable for parenteral or pulmonary delivery.

In accordance with one aspect of the present invention, the composition further includes mesenchymal stromal cell-derived extracellular vesicles.

In accordance with one aspect of the present invention, the composition is co-administered with an antiviral compound selected from oseltamivir, zanamivir, or baloxavir.

In accordance with one aspect of the present invention, the present invention provides a pharmaceutical composition, which includes a lactoferrin present at a concentration of approximately 0.01% to 10% (w/v), and a pharmaceutically acceptable carrier suitable for intravenous, intranasal, oral, or inhalable delivery. The lactoferrin is identified through proteomic analysis as a functional protein enriched in extracellular vesicles derived from umbilical cord-derived mesenchymal stromal cells (UC-MSCs). The composition is formulated for intravenous, intranasal, or inhalable administration in the treatment of acute lung injury caused by a respiratory viral infection.

In accordance with one aspect of the present invention, the lactoferrin is selected from recombinant human lactoferrin or purified bovine lactoferrin.

In accordance with one aspect of the present invention, the pharmaceutical composition further including one or more excipients selected from surfactants, isotonic agents, buffers, or viscosity modifiers.

In accordance with one aspect of the present invention, the pharmaceutical composition is packaged in a unit-dose vial, a nebulizer cartridge, or a nasal spray bottle.

In accordance with one aspect of the present invention, the lactoferrin is solubilized in phosphate-buffered saline or a citrate buffer.

In accordance with one aspect of the present invention, the pharmaceutical composition is lyophilized and reconstituted prior to administration.

In accordance with one aspect of the present invention, the pharmaceutical composition further includes an antiviral drug selected from zanamivir, oseltamivir phosphate or baloxavir marboxil.

In accordance with one aspect of the present invention, the respiratory virus includes a highly pathogenic avian influenza virus.

To demonstrate therapeutic efficacy, the invention utilizes a physiologically relevant in vitro human lung injury model and a murine influenza A (H5N1) virus infection model. In both models, LTF alone was shown to restore alveolar epithelial barrier function and reduce proinflammatory cytokines, with performance comparable to or better than MSC-EVs. This highlights the feasibility of using LTF as a standalone agent.

By isolating a specific protein component from the MSC-EV secretome, the invention circumvents challenges associated with the expansion, storage, and administration of living stromal cells. The cell-free approach eliminates variability and enhances scalability while maintaining the desired anti-inflammatory and antiviral effects. Compared to current treatments and MSC-based products, the invention provides a novel application of a pharmaceutically acceptable and FDA-approved supplement for respiratory disease intervention. It represents a significant advancement over the prior art by offering a clinically accessible, well-tolerated, and mechanism-supported strategy for the treatment of viral-induced ALI.

### BRIEF DESCRIPTION OF THE DRAWINGS

Embodiments of the invention are described in more details hereinafter with reference to the drawings, in which:

FIG. 1A shows the experimental setup in which human alveolar epithelial cells (AECs) cultured in transwell inserts were infected with influenza A (H5N1) virus to monitor changes in alveolar fluid clearance (AFC) and alveolar protein permeability (APP). FIG. 1B shows that MSC-derived extracellular vesicle (MSC-EV) treatment of H5N1-infected AECs restored the impaired AFC compared to untreated infected cells. FIG. 1C shows that MSC-EV treatment significantly decreased the APP caused by H5N1 infection. FIG. 1D shows the classification of cellular components associated with proteins identified in MSC-EVs by proteomic analysis. FIG. 1E shows the biological processes enriched among the proteins carried by MSC-EVs, as revealed by proteomic profiling;

FIG. 2A shows a schematic representation of the in vitro lung injury model used to evaluate the therapeutic effects of MSC-EVs and LTF on human AECs infected with H5N1 virus. FIG. 2B shows that both MSC-EV and LTF treatments attenuated the H5N1-induced impairment of AFC in infected AECs. FIG. 2C shows that both MSC-EV and LTF treatments reduced the increased APP caused by H5N1 infection. FIG. 2D shows that both MSC-EV and LTF treatments exerted anti-inflammatory effects on H5N1-infected AECs, as indicated by reduced levels of proinflammatory cytokines. FIG. 2E shows that MSC-EV and LTF treatments inhibited the attachment and internalization of H5N1 virus particles into AECs. FIG. 2F shows that both treatments upregulated the expression of anti-viral genes in infected AECs. FIG. 2G shows that MSC-EV and LTF treatments significantly suppressed the replication of H5N1 virus, as evidenced by reduced expression of the influenza viral M-gene;

FIG. 3A shows a schematic diagram of the in vivo experimental protocol, wherein mice were intranasally infected with H5N1 influenza virus followed by intravenous administration of MSC-EVs or LTF as treatment. FIG. 3B shows that body weight loss among H5N1-infected mice was not significantly different between the treated and untreated groups. FIG. 3C shows that survival rates of H5N1-infected mice were improved following treatment with either MSC-EVs or LTF. FIG. 3D shows that pro-inflammatory cytokine levels in lung homogenates were significantly reduced in mice treated with MSC-EVs or LTF. FIG. 3E shows that pro-inflammatory cytokine levels in bronchoalveolar lavage (BAL) fluid were significantly suppressed by MSC-EV or LTF treatment. FIG. 3F shows that expression levels of the influenza viral M-gene were decreased in the lungs of treated mice, indicating a reduction in viral load. FIG. 3G shows that the tissue culture infectious dose (TCID) assay confirmed comparable antiviral effects of MSC-EV and LTF treatments in vivo; and

FIG. 4A shows that body weight loss among H5N1-infected mice was not significantly different between the treated and untreated groups. FIG. 4B shows that survival rates of H5N1-infected mice were improved following treatment with LTF or zanamivir, but not when in combination. FIG. 4C shows that pro-inflammatory cytokine levels in lung homogenates were significantly reduced in mice treated with LTF, zanamivir or in combination. FIG. 4D shows that the tissue culture infectious dose (TCID) assay confirmed potent antiviral effect of LTF, zanamivir and in combination in vivo.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the term “acute lung injury” (ALI) refers to a clinical syndrome characterized by diffuse alveolar damage, disruption of the alveolar-capillary barrier, pulmonary edema, impaired gas exchange, and an inflammatory response in the lungs. ALI may be caused by infectious agents such as respiratory viruses, including highly pathogenic avian influenza viruses, and is considered a precursor to acute respiratory distress syndrome (ARDS).

As used herein, the term “alveolar fluid clearance” (AFC) refers to the net transport of fluid from the alveolar space to the interstitium and bloodstream, primarily mediated by active ion transport across the alveolar epithelium. An increase in AFC indicates improved epithelial function and resolution of pulmonary edema. In vitro, AFC may be measured based on the dilution of FITC-dextran over time in transwell culture systems.

As used herein, the term “alveolar epithelial protein permeability” (APP) refers to the extent to which macromolecules, such as proteins, can traverse the alveolar epithelial barrier. An increase in APP indicates disruption of tight junction integrity and barrier dysfunction. In vitro, APP is measured by quantifying the translocation of fluorescently labeled dextran from the apical to basolateral compartment across a monolayer of alveolar epithelial cells.

Lactoferrin used in the present invention is a naturally occurring iron-binding glycoprotein having a molecular weight of approximately 80 kDa and comprising approximately 700 amino acid residues. The lactoferrin may be derived from human or bovine sources and may be produced via recombinant expression systems. Commercially available preparations (e.g., recombinant human lactoferrin from rice or yeast expression systems) may be used.

As used herein, the term “mesenchymal stromal cell-derived extracellular vesicles” (MSC-EVs) refers to nanoscale membrane-bound vesicles secreted by mesenchymal stromal cells. MSC-EVs contain a variety of bioactive molecules, including proteins, lipids, and RNAs, and mediate intercellular communication. In the context of this invention, MSC-EVs may be derived from umbilical cord-derived MSCs and isolated using ultracentrifugation or equivalent techniques.

As used herein, the term “highly pathogenic avian influenza virus” refers to a class of influenza A viruses that cause severe disease and high mortality rates in birds and humans. Examples include H5N1 and H7N9 subtypes. In this invention, the representative virus includes A/Hong Kong/483/97 and A/Hong Kong/486/1997 strains of H5N1.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

Highly pathogenic avian influenza viruses, such as the H5N1 subtype, are known to cause severe lower respiratory tract infections and are considered a significant threat to global public health. Conventional antiviral treatments, such as oseltamivir, have shown limited efficacy in reducing mortality associated with H5N1 infection. Severe respiratory virus infections frequently progress to acute lung injury (ALI), or its more advanced form, acute respiratory distress syndrome (ARDS), which are characterized by pulmonary inflammation, disruption of the alveolar-capillary barrier, and impaired fluid homeostasis in the lungs. Clinical and preclinical studies have demonstrated that H5N1 virus infection can directly impair AFC and increase APP, both of which are key pathological features of ALI. Restoration of AFC and stabilization of barrier function are considered essential for mitigating disease progression. Although a variety of pharmacologic interventions have been explored in clinical trials for the treatment of ARDS, to date no effective drug-based therapy has been approved.

Cell-based therapies have recently gained attention as a promising alternative for treating ALI and other inflammatory lung disorders. In particular, mesenchymal stromal cells (MSCs) and their secreted extracellular vesicles (EVs) have demonstrated therapeutic effects due to their immunomodulatory, anti-inflammatory, and regenerative properties. EVs are nanoscale vesicles released by live cells, capable of mediating intercellular communication via transfer of bioactive proteins, RNAs, and other molecular cargo. Among them, MSC-EVs have been reported to suppress inflammation, promote tissue repair, and modulate immune responses. Despite the expanding body of research on MSC-EVs, limited studies have investigated their efficacy against highly pathogenic respiratory viral infections. Moreover, the complexity of MSC-EV production and the heterogeneity of vesicle content present logistical and regulatory challenges for clinical translation.

LTF is a naturally occurring iron-binding glycoprotein that has been widely studied for its antimicrobial, antiviral, and immunomodulatory functions. Originally discovered in bovine milk and later identified in human secretions, LTF is synthesized by exocrine glands and plays an important role in innate immune defense. It acts by sequestering iron, thereby limiting microbial growth, and by modulating immune responses at mucosal surfaces. Notably, human and bovine LTF share high sequence homology (approximately 69%) and exhibit comparable biological activity, which has led to extensive preclinical use of the bovine variant. LTF has been granted “generally recognized as safe” (GRAS) status by the U.S. Food and Drug Administration and is approved for use as a dietary supplement in both the United States and Europe. Despite its well-established safety and multifunctional bioactivity, the role of LTF in combating H5N1-induced lung injury remains unexplored.

The pathophysiology of H5N1 infection includes activation of proinflammatory cytokine cascades that disrupt epithelial integrity and impair ion transporter function, leading to fluid accumulation in the alveolar space.

To investigate this mechanism, a physiologically relevant in vitro lung injury model was employed in the present invention, consisting of primary human AECs cultured on transwell membranes and infected with H5N1 virus. This model enables quantitative assessment of virus-induced changes in AFC, APP, and inflammatory responses.

In the present invention, it was discovered that treatment with either MSC-EVs or recombinant human LTF restored impaired AFC and reduced epithelial permeability in H5N1-infected AECs. Both treatments also downregulated the expression of key proinflammatory cytokines and significantly reduced viral replication. Proteomic profiling of MSC-EVs identified LTF as one of the most abundant protein cargos, implicating it as a major contributor to their observed bioactivity. In vivo studies using a murine model further confirmed the therapeutic potential of LTF, which demonstrated anti-inflammatory and antiviral effects comparable to those of MSC-EVs, along with improved survival outcomes. Caution should be taken when administering LTF in combination with existing antivirals, e.g. zanamivir, as LTF is shown to outperform zanamivir in increasing survival rate and the suppression of H5N1-dysregulated pro-inflammatory cytokines, however, when administered in combination, unexpected effects may appear. These findings support the application of LTF as a standalone, cell-free therapeutic agent for the treatment of influenza-induced acute lung injury.

In one embodiment, the invention provides a method for treating virus-induced acute lung injury comprising the administration of a therapeutically effective amount of lactoferrin to a subject in need thereof, wherein the lactoferrin restores alveolar fluid clearance and reduces alveolar protein leakage.

In one embodiment, the lactoferrin is derived from a bovine or human source and may be in the form of a recombinant protein or a purified natural product.

In one embodiment, the lactoferrin is administered at a dose ranging from about 100 mg/kg to about 1000 mg/kg body weight per day, preferably from about 100 mg/kg to about 500 mg/kg, depending on the subject's condition, route of administration, and disease severity.

In one embodiment, the lactoferrin is formulated as a pharmaceutical composition selected from a group consisting of a sterile injectable solution, an inhalable powder, a nasal spray, a nebulized suspension, or an oral capsule or tablet.

In one embodiment, the method further comprises reducing the expression of proinflammatory cytokines selected from the group consisting of MCP-1, IL-6, IL-8, RANTES, and TNF-α in infected alveolar epithelial cells.

In one embodiment, the method comprises inhibiting the attachment and internalization of respiratory viruses into alveolar epithelial cells via administration of lactoferrin.

In one embodiment, the therapeutic agent is formulated for systemic administration, such as intravenous injection, or for local delivery via inhalation or intranasal instillation.

In one embodiment, the method is applied in conjunction with existing antiviral agents or supportive care, thereby enhancing overall treatment outcomes in patients suffering from ALI or ARDS.

In one embodiment, the pharmaceutical composition comprises lactoferrin in combination with one or more pharmaceutically acceptable excipients, stabilizers, or delivery agents suitable for parenteral, intranasal, or pulmonary administration.

In one embodiment, the lactoferrin is administered in combination with one or more therapeutic agents selected from antiviral agents, anti-inflammatory agents, corticosteroids, cytokine inhibitors, or MSC-EVs.

In another aspect, the invention provides a combination therapy comprising lactoferrin and an antiviral agent, wherein co-administration results in enhanced suppression of viral replication and improved survival outcomes.

In summary, the present invention provides a method for treating acute lung injury caused by respiratory viral infections, including but not limited to highly pathogenic H5N1, using lactoferrin as a therapeutic agent. By directly targeting viral attachment, epithelial barrier dysfunction, and cytokine-mediated inflammation, LTF offers a novel therapeutic mechanism distinct from conventional antivirals or immunosuppressants. The invention further suggests that specific bioactive proteins derived from MSC-EVs, such as LTF, can be isolated and formulated for clinical use, thereby circumventing the complexity and risks associated with cell-based therapies.

### EXAMPLE

### Example 1—Materials and Methods

### Virus

H5N1 was used for in vitro infection. All influenza viruses were passaged in Madin-Darby Canine Kidney (MDCK) cells. Viral titers were determined by median tissue culture infectious dose (TCID₅₀). All experiments were performed inside a biosafety level-3 facility.

### Culture of UC-MSCs

Umbilical Cord-MSCs were isolated by HealthBaby Biotech (Hong Kong) Company Ltd, and cultured in low glucose (1.0 g/L) Dulbecco's modified Eagle's medium (DMEM) (Gibco, USA) supplemented with 10% HyClone fetal bovine serum (FBS) (SV30160.03, Thermo Fisher) and 1% P/S was used for cell culture. UC-MSCs were cultured in humidified atmosphere (37° C., 5% CO2) with growth medium changed every 48-72 hours. Cells were trypsinized and seeded for experimental use upon 70% confluency.

Isolation of EVs from UC-MSCs

At 70% confluency, UC-MSCs were washed 3 times with HBSS to remove HyClone FBS, then replenished with 10% EV-depleted FBS low glucose DMEM. Supernatant was collected at 72 h and stored at −20° C. until EV isolation. Differential centrifugation was employed to isolate EVs with the following steps: 1) centrifugation at 3,000 rpm for 15 minutes at 4° C.; 2) harvest supernatant and centrifuge again at 20,000 g for 30 minutes at 4° C.; 3) harvest supernatant, pass it through a 0.22 μM filter, and centrifuge at 100,000 g for 2 hours at 4° C.; 4) discard supernatant, wash EV pellet with PBS and centrifuge EV suspension for another 2 hours at 100,000 g at 4° C.; 5) remove supernatant, resuspend EV pellet in PBS and store at −20° C. until use.

### Isolation and Culture of Primary Human AECs

AECs were isolated from resected, non-malignant lung tissue obtained from the Department of the Cardiothoracic Surgery, Queen Mary Hospital, Hong Kong. Firstly, visible bronchi were removed and lung tissue was subject to mincing into pieces of >0.5 mm thickness using scissors. Minced lung pieces were washed with Hank's balanced salt solution at pH 7.4 (Invitrogen, USA) to partially remove macrophages and blood cells. Washed lung pieces were then digested using 0.5% trypsin (GibcoBRL, USA) and 4 U/ml elastase (Worthington Biochemical, USA) for 40 minutes (mins) at 37° C. in a shaking water-bath. Digestion was terminated with 40% FBS DMEM/F12 medium and DNase I (350 units/ml) (Sigma, USA). Incompletely digested lung was separated using a cell strainer of 50 μM pore size (BD Bioscience, USA). Cell clumps were dispersed by repeatedly pipetting for 10 mins. After spinning down, cells were incubated with a 1:1 mixture of DMEM/F12 medium and small airway growth medium (SAGM) (Lonza, USA) containing 5% FBS and 350 units/ml DNase I. Cells were seeded on a new tissue culture flask (Corning, USA) for adhesion at 37° C. Cells that did not adhere were collected for centrifugation and resuspended with SAGM in a new tissue culture flask. Culture medium was changed daily within the first 4 days of plating. AECs were trypsinized for seeding upon 75% confluency.

### In Vitro Acute Lung Injury Model

To study the effect of influenza viruses on alveolar fluid clearance and protein permeability in human alveolar epithelial cells, a physiologically relevant 24-transwell in vitro acute lung injury model was used. The alveolar epithelial cells were plated on the apical chamber of 24-well Costar Transwell inserts with a 0.4-μm pore size (Corning), at a density of 1×10⁵ cells per well. The microporous transwell membrane established a liquid-liquid interface similar to that of human lung epithelium, and the plated cells were maintained in a humidified atmosphere (5% CO₂, 37° C.). Transepithelial resistance was maintained at ≥800 Ω/cm², which indicates good tight junction integrity between cells.

### Measurement of Alveolar Fluid Clearance and Alveolar Protein Permeability

Net alveolar fluid transport from the apical chamber of the transwell culture insert (containing a monolayer of alveolar epithelial cells infected with influenza virus) to the basolateral chamber of the transwell at 24 hours post infection was measured. Alveolar epithelial cells were inoculated with the respective influenza A viruses at a multiplicity of infection (MOI) of 0.1 for 1 hour. Then, 200 μl of FITC-labeled 70 kDa dextran (Sigma) was added to the cells (final concentration, 500 ng/μl), along with the addition of either 1×10⁸ MSC-EVs or 100 μg/ml human recombinant LTF to the apical media for 24 h treatment. After 5 minutes, a sample of 100 μl was collected from the apical chamber for initial FITC measurement, after which it is transferred back into the apical chamber for overnight incubation. After 24 hours of dextran incubation, 100 μl was collected from the apical chamber, while 100 μl was collected from the basolateral chamber for the final FITC measurement. The fluorescence of each sample was measured by a modulus fluorometer (FLUOstar OPTIMA, BMG Labtech) at excitation wavelength of 485 nm and emission wavelength of 520 nm. A standard curve of known FITC concentration was constructed for the calculation of FITC-dextran present in the transwell chambers at different time points. The net alveolar fluid transport across the epithelial monolayer was measured as [1−(FITC concentration in the initial apical sample/FITC concentration in the final apical sample)]/200 μl/0.33 cm²/24 hours. The final basolateral reading was collected after 24 hours of dextran incubation to measure protein permeability, based on the unidirectional flux of fluorescence-labeled dextran from the apical to the basolateral chamber of the transwell culture insert.

### Virus Infection of Mice

6-8 weeks old female BALB/c mice were intranasally inoculated with 10⁶ log TCID₅₀ of A/Hong Kong/486/1997 (H5N1) influenza virus in 25 μl volume. On days 3, 5 and 7 post infection, mice were injected intravenously with 1×10⁸ MSC-EVs, 100 μg/ml human recombinant LTF, 10 mg/kg zanamivir, or PBS in 100 μl. Survival and body weight were monitored for 10 days. On day 10 post infection, virus was titrated in three mouse lungs per group. BAL fluid was collected for cytokine assay using a mouse Luminex assay according to the manufacturer's instruction manual (R&D Systems, USA). Also, three mouse lungs per group were fixed for histopathological analysis.

Quantification of Influenza Matrix Gene, Inflammatory Cytokines, Anti-Viral Genes and Ion Transporter mRNA by Quantitative RT-PCR

Total RNA and cellular protein were extracted from infected AEC at 24 hours post infection. RNA extraction was performed, and the RNA was then reverse transcribed into complementary DNA using PrimeScript RT Reagent Kit (TaKaRa, Dalian), both according to manufacturer's instructions. AECs infected with influenza viruses at MOI 2 were lysed with 350 μl buffer RLT (Qiagen, Germany) with beta-mercaptoethanol (Sigma, USA). Extraction of RNA was carried out using the MiniBEST Universal RNA extraction kit (TaKaRa, Dalian) with DNase treatment as per manufacturer's instructions. PrimeScript RT reagent kit (TaKaRa, China) was used for reverse transcription. ViiA™ 7 Real-Time PCR System (Applied Biosystem, USA) was used to perform real-time PCR. Gene expression profiles were normalized with housekeeping gene β-actin mRNA. Standard plasmid with a known copy number was run in conjunction with gene of study for the generation of a standard curve to determine absolute copy numbers. Master mix including Power SYBR Green PCR Master Mix (Applied Biosystem, USA) with cDNA reverse-transcribed from 500 ng of total RNA, were amplified by real-time PCR for 40 cycles with an ABI 7500 PCR system (Applied Biosystem, USA). Gene expression and statistical analysis were calculated following the instructions provided by the manufacturer.

### Statistical Analysis

All in vitro experiments were conducted independently in triplicates. Groups were compared by using an unpaired, two-tailed t test. Differences were considered significant if P≤0.05.

### Example 2—Evaluation of MSC-EV-Mediated Protection Against H5N1-Induced Epithelial Barrier Dysfunction

In this example, the therapeutic efficacy of MSC-EVs in mitigating H5N1-induced alveolar epithelial barrier dysfunction was evaluated using a physiologically relevant in vitro lung injury model.

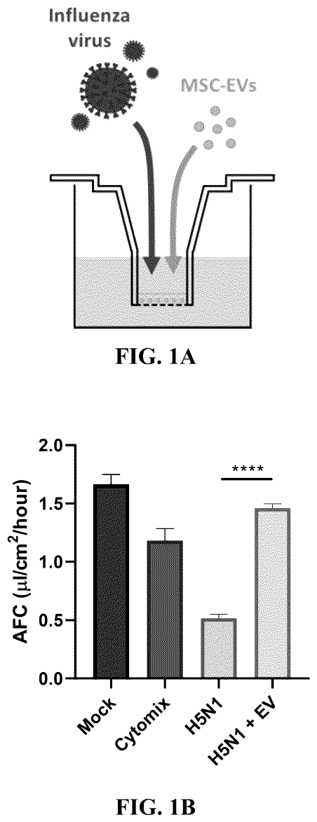

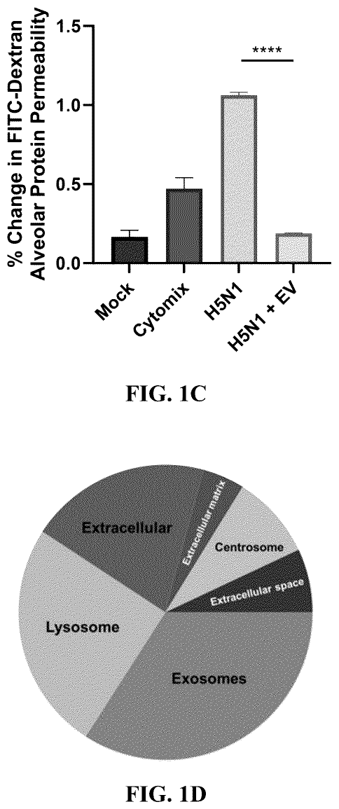

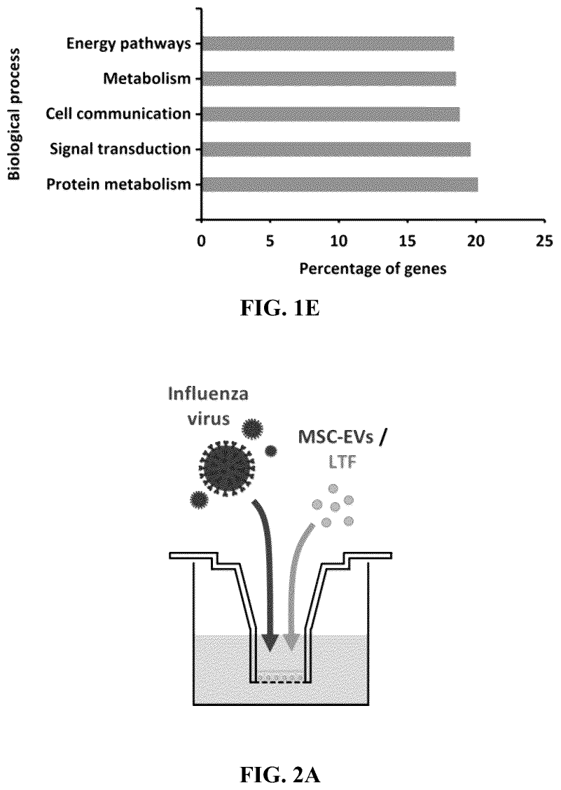

Primary human AECs were seeded on transwell inserts to establish a tight epithelial monolayer under air-liquid interface conditions. Upon reaching confluency, the apical chamber was inoculated with influenza A/HK/483/97 (H5N1) virus at a multiplicity of infection (MOI) of 0.1. As schematically illustrated in FIG. 1A, virus and MSC-EVs were administered through the apical compartment. A control group was treated with a cytokine mixture (Cytomix: IL-1B, TNF-α, and IFN-γ) to induce barrier damage.

After 24 hours of H5N1 infection, a significant reduction in AFC and increase in APP were observed, confirming disruption of epithelial barrier function. As shown in FIG. 1B, H5N1-infected AECs exhibited a drastic decline in AFC (˜0.5 μL/cm²/hour) compared to mock-infected controls (˜1.5 L/cm²/hour), while MSC-EV treatment significantly restored AFC to near-baseline levels (P<0.0001).

Similarly, FITC-dextran leakage assays demonstrated that MSC-EVs reduced the elevated protein permeability induced by H5N1. As depicted in FIG. 1C, the percentage increase in FITC-dextran translocation across the epithelial layer was markedly elevated in H5N1-infected cells (˜1.0 arbitrary units) but significantly suppressed following MSC-EV treatment (˜0.2 units), indicating preserved tight junction integrity.

To identify functional protein components responsible for the therapeutic effect, proteomic analysis was conducted on isolated MSC-EVs. Subcellular classification revealed that the majority of EV cargo proteins localized to exosomes, lysosomes, and extracellular compartments (FIG. 1D), consistent with their secretory and signaling functions. Functional enrichment analysis further showed that the EV proteins were predominantly involved in protein metabolism, signal transduction, and cell communication pathways (FIG. 1E), all of which are associated with tissue repair and antiviral response.

Table 1 shows the top five most abundantly expressed proteins identified in MSC-EVs, with LTF highlighted as a key component contributing to the observed therapeutic effects. Out of the top 5 proteins expressed in MSC-EVs, “Lactotransferrin” is of particular interest due to their reported antiviral and anti-inflammatory properties in other diseases.

TABLE 1

| Protein | Function |
|---|---|
| Lactoferrin | Iron binding, antiviral, anti-inflammatory |
| Action, cytoplasmic 2 | Cell motility, cytoskeleton maintenance |
| Albumin | Protein transport |
| Decorin | Connective tissue component, matrix assembly |
| Alpha-2-macroglobulin | Broad-spectrum protease inhibitor |

Collectively, these findings demonstrate that MSC-EVs exert barrier-protective and immunomodulatory effects in H5N1-infected alveolar epithelial cells. In particular, the identification of lactoferrin as a major active cargo supports the feasibility of employing selected MSC-EV-derived proteins in place of whole EV preparations for treating virus-induced acute lung injury.

### Example 3—Therapeutic Effect of Lactoferrin in H5N1-Infected Alveolar Epithelium In Vitro

This example investigates the therapeutic efficacy of human recombinant LTF, a major functional protein found in MSC-EVs, in attenuating H5N1-induced lung epithelial injury using an in vitro lung injury model.

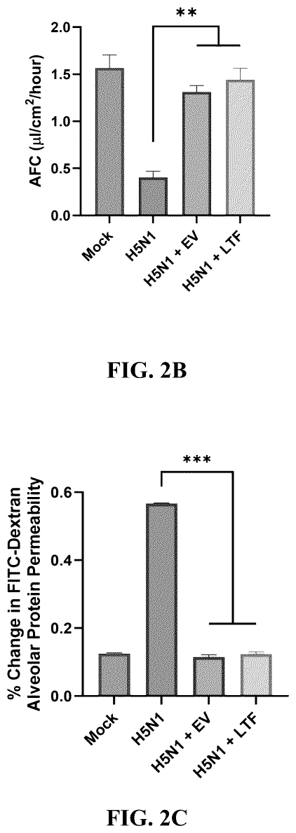

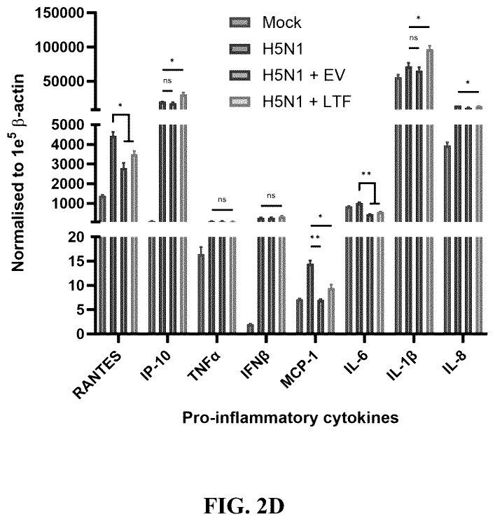

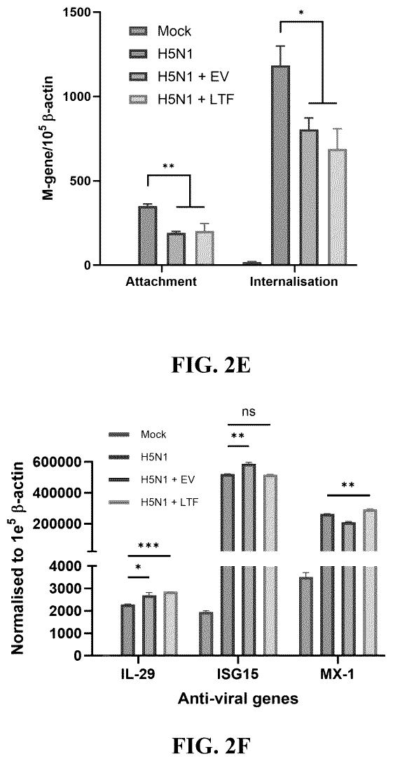

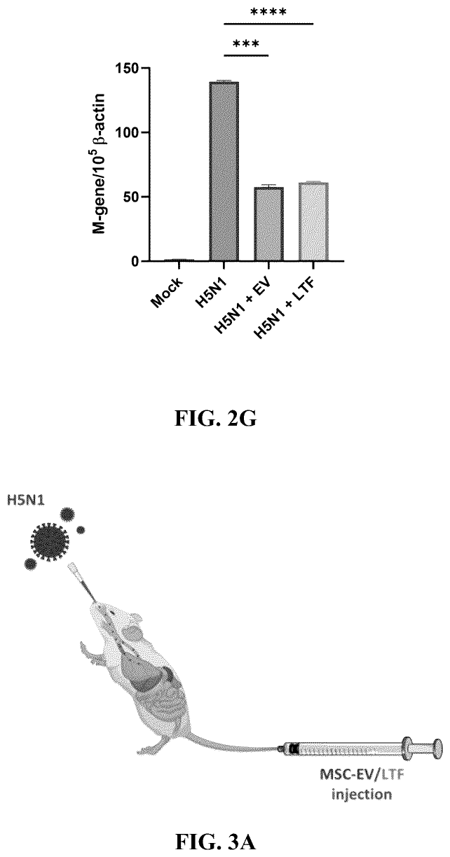

As schematically illustrated in FIG. 2A, primary human AECs were cultured on transwell inserts and infected with influenza A/HK/483/97 (H5N1) virus (MOI=0.1). Upon infection, the cells were treated with either MSC-EVs (1×10⁸ particles per well) or recombinant human lactoferrin (100 μg/mL) for 24 hours.

As shown in FIG. 2B, H5N1 infection caused a significant decrease in AFC, while treatment with either MSC-EVs or LTF partially restored the impaired AFC. Likewise, FIG. 2C demonstrates that both treatments significantly reduced the increased APP caused by viral infection, indicating protection of epithelial tight junction integrity.

The anti-inflammatory effects of MSC-EVs and LTF were evaluated by quantifying cytokine secretion in the culture supernatants. As depicted in FIG. 2D, LTF treatment significantly suppressed the secretion of RANTES, MCP-1, IL-6, and IL-8 in infected AECs, although the magnitude of suppression was generally lower than that observed with MSC-EV treatment. These findings confirm the immunomodulatory effect of LTF.

To evaluate antiviral activity, the ability of LTF to interfere with viral entry was assessed. As shown in FIG. 2E, both MSC-EVs and LTF significantly reduced the attachment and internalization of H5N1 virus particles into AECs. Moreover, treatment with LTF led to a marked upregulation of endogenous antiviral gene expression (e.g., IFITM3, MX1), as shown in FIG. 2F, suggesting activation of host antiviral defense pathways.

Consistent with these findings, viral replication was significantly suppressed by LTF treatment, as measured by influenza M-gene expression levels (FIG. 2G). The reduction in viral RNA levels was comparable to that observed with MSC-EV treatment, demonstrating that LTF alone possesses antiviral efficacy in the infected lung epithelial model.

Taken together, these results indicate that lactoferrin exerts multiple protective effects, including the restoration of alveolar fluid clearance, the maintenance of epithelial barrier function, the suppression of inflammatory cytokines, and the inhibition of viral replication in H5N1-infected human alveolar epithelial cells. The therapeutic efficacy observed for lactoferrin, which is comparable to that of MSC-derived extracellular vesicles, supports its application as a standalone, cell-free therapeutic candidate for the treatment of viral-induced acute lung injury.

### Example 4—In Vivo Evaluation of MSC-EV and Lactoferrin Therapeutic Activity Against H5N1-Induced Lung Injury

This example evaluates the therapeutic potential of MSC-EVs and recombinant human LTF in vivo, using a murine model of acute lung injury induced by H5N1 virus.

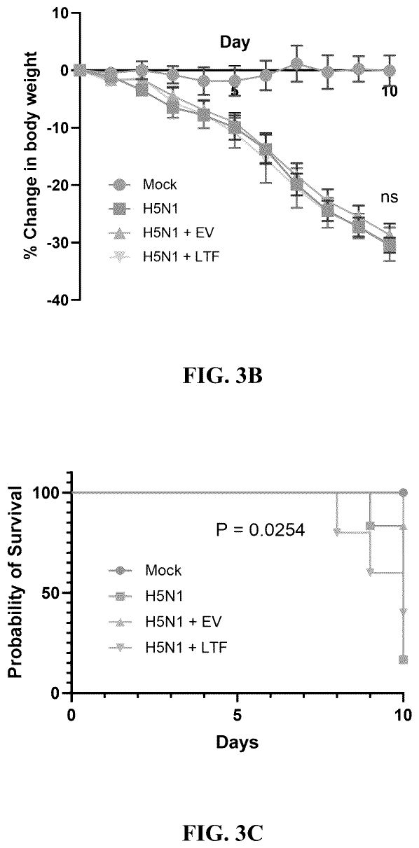

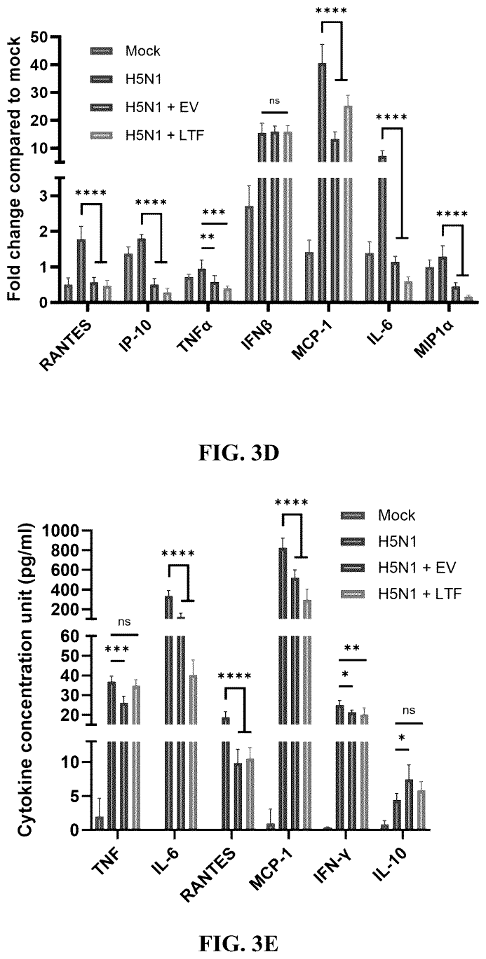

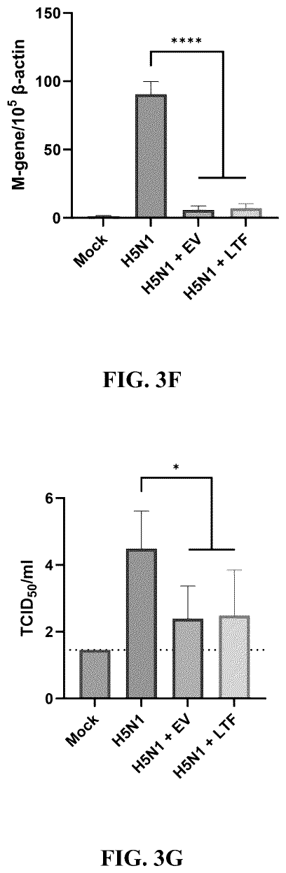

Female BALB/c mice (6-8 weeks old) were intranasally infected with 10⁶ TCID₅₀ of H5N1 (A/HK/486/1997) virus. Upon onset of clinical symptoms, mice were intravenously administered either MSC-EVs (1×10⁸ particles in 100 μL PBS) or LTF (100 μg in 100 μL PBS) on days 3, 5, and 7 post-infection, as shown in FIG. 3A. Mice receiving only PBS served as the untreated control group.

As illustrated in FIG. 3B, no statistically significant difference in body weight loss was observed among the treated and untreated groups over the 10-day monitoring period, indicating that the treatment did not alter overall weight dynamics under infection conditions. However, survival analysis in FIG. 3C demonstrated that mice treated with MSC-EVs exhibited a significantly increased survival rate compared to untreated controls, while LTF-treated mice also showed a modest improvement in survival.

Proinflammatory cytokines were measured in lung homogenates and BAL fluid using multiplex Luminex assays. As shown in FIG. 3D and FIG. 3E, both MSC-EV and LTF treatments resulted in significant reduction of inflammatory mediators. Specifically, MSC-EVs suppressed a broad spectrum of cytokines including IL-6, MCP-1, and TNF-α, while LTF showed a more pronounced effect in reducing MCP-1, IL-6, and MIP-1α levels, suggesting potent anti-inflammatory activity.

Viral replication was assessed by quantifying the expression of the influenza M-gene in lung tissue samples. As depicted in FIG. 3F, both MSC-EVs and LTF significantly reduced viral gene expression levels compared to untreated mice. In addition, the TCID assay shown in FIG. 3G confirmed that viral titers were markedly suppressed by both treatment groups, indicating comparable antiviral efficacy.

### Example 5—Combination Therapy of LTF and Zanamivir in H5N1-Infected Mice

To examine whether combinational treatment of LTF with an existing antiviral can further improve therapeutic potential, the same murine model of acute lung injury induced by H5N1 virus was used as described in Example 4.

Female BALB/c mice (6-8 weeks old) were intranasally infected with 10⁶ TCID₅₀ of H5N1 (A/HK/486/1997) virus. Upon onset of clinical symptoms, mice were intravenously administered either LTF (100 μg in 100 μL PBS), zanamivir (10 mg/kg body weight), or in combination on days 3, 5, and 7 post-infection. Mice receiving only PBS served as the untreated control group.

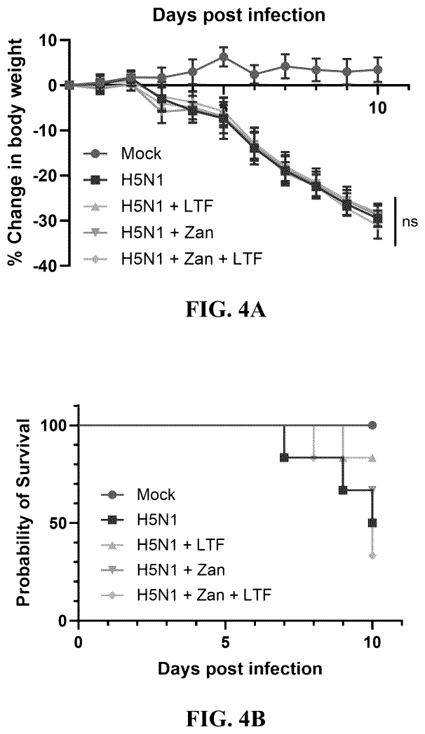

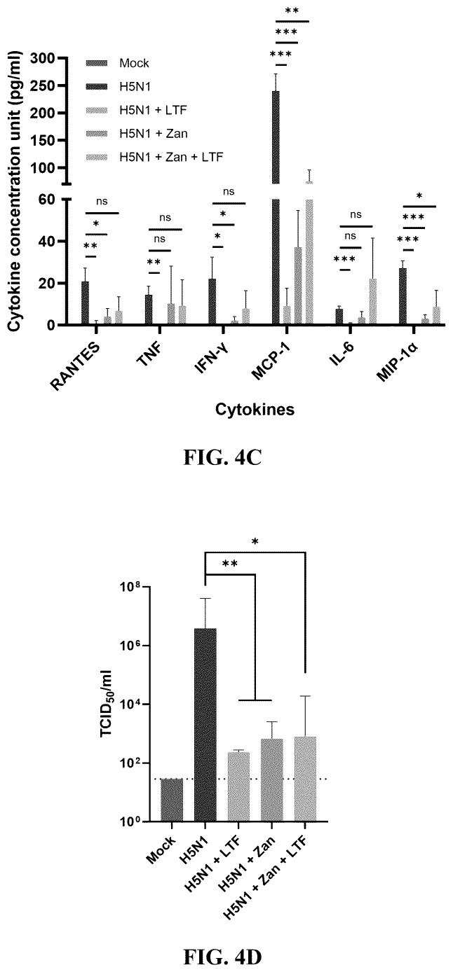

As illustrated in FIG. 4A, no statistically significant difference in body weight loss was observed among the treated and untreated groups over the 10-day monitoring period, indicating that the treatment did not alter overall weight dynamics under infection conditions. However, survival analysis in FIG. 4B demonstrated that mice treated with LTF exhibited a significantly increased survival rate compared to untreated controls, while zanamivir-treated mice also showed a modest improvement in survival.

Proinflammatory cytokines was measured in BAL fluid using multiplex Luminex assays. As shown in FIG. 4C, all three treatments resulted in significant reduction of inflammatory mediators. Specifically, LTF suppressed a broad spectrum of cytokines including RANTES, TNF, IFN-γ, MCP-1, IL-6, and MIP-1α, with the other two treatments exhibiting less pro-inflammatory activities.

Viral replication in lung tissue samples was assessed by TCID assay. As depicted in FIG. 4D, viral titers were markedly suppressed by all treatment groups, indicating comparable antiviral efficacy.

These in vivo results collectively support the therapeutic benefits of both MSC-EVs and LTF in mitigating H5N1-induced lung pathology. The treatments reduced viral burden and inflammatory cytokine levels, leading to improved survival outcomes. Notably, LTF demonstrated an anti-inflammatory profile distinct from, and in some parameters superior to, MSC-EVs, which further supports its feasibility as a standalone, cell-free therapeutic candidate for respiratory viral infections.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A method of treating acute lung injury in a subject infected with a respiratory virus, comprising administering to the subject a composition comprising lactoferrin identified as a functional protein enriched in extracellular vesicles derived from umbilical cord-derived mesenchymal stromal cells (UC-MSC-EVs), and wherein the administration increases alveolar fluid clearance by at least 50% and reduces alveolar epithelial protein permeability by at least 50% relative to a non-treated control.

2. The method of claim 1, wherein the respiratory virus comprises a highly pathogenic avian influenza virus.

3. The method of claim 2, wherein the highly pathogenic avian influenza virus comprises A/HK/483/97 or A/HK/486/1997 H5N1 virus.

4. The method of claim 1, wherein the composition comprises lactoferrin at a dose of about 100 to 1000 mg/kg body weight per day.

5. The method of claim 1, wherein the lactoferrin is selected from recombinant human lactoferrin or purified bovine lactoferrin.

6. The method of claim 1, wherein the composition is administered intravenously, intranasally, orally, or by inhalation.

7. The method of claim 1, wherein the administration reduces expression of one or more cytokines selected from the group consisting of MCP-1, IL-6, IL-8, RANTES, TNF-α, and MIP-1α.

8. The method of claim 1, wherein the composition inhibits attachment or internalization of the virus into alveolar epithelial cells.

9. The method of claim 1, wherein the composition suppresses expression of the influenza viral matrix (M) gene.

10. The method of claim 1, wherein the lactoferrin is formulated with one or more excipients selected from stabilizers, surfactants, or buffers suitable for parenteral or pulmonary delivery.

11. The method of claim 10, wherein the composition further comprises mesenchymal stromal cell-derived extracellular vesicles.

12. The method of claim 1, wherein the composition is co-administered with an antiviral compound selected from oseltamivir, zanamivir, or baloxavir.

13. A pharmaceutical composition, comprising:
a lactoferrin present at a concentration of approximately 0.01 to 10% (w/v), wherein the lactoferrin is identified through proteomic analysis as a functional protein enriched in extracellular vesicles derived from umbilical cord-derived mesenchymal stromal cells (UC-MSCs); and
a pharmaceutically acceptable carrier suitable for intravenous, intranasal, oral, or inhalable delivery,
wherein the composition is formulated for intravenous, intranasal, oral, or inhalable administration in the treatment of acute lung injury caused by a respiratory viral infection.

14. The pharmaceutical composition of claim 13, wherein the lactoferrin is selected from recombinant human lactoferrin or purified bovine lactoferrin.

15. The pharmaceutical composition of claim 13, wherein the pharmaceutical composition further comprising one or more excipients selected from surfactants, isotonic agents, buffers, or viscosity modifiers.

16. The pharmaceutical composition of claim 13, wherein the pharmaceutical composition is packaged in a unit-dose vial, a nebulizer cartridge, or a nasal spray bottle.

17. The pharmaceutical composition of claim 13, wherein the lactoferrin is solubilized in phosphate-buffered saline or a citrate buffer.

18. The pharmaceutical composition of claim 13, wherein the pharmaceutical composition is lyophilized and reconstituted prior to administration.

19. The pharmaceutical composition of claim 13, wherein the pharmaceutical composition further comprises an antiviral drug selected from zanamivir, oseltamivir phosphate or baloxavir marboxil.

20. The pharmaceutical composition of claim 13, wherein the respiratory virus comprises a highly pathogenic avian influenza virus.
